(19) United States
(12) Patent Application Publication
(10) Pub. No.: US 2015/0051636 A1
(43) Pub. Date: Feb. 19, 2015

(54) SIMPLIFIED NASAL DILATOR

(71) Applicant: Hanford N. Lockwood, JR., San Mateo, CA (US)

(72) Inventor: Hanford N. Lockwood, JR., San Mateo, CA (US)

(21) Appl. No.: 13/966,174

(22) Filed: Aug. 13, 2013

(51) Int. Cl. A61F 5/08; A61M 29/00

(52) U.S. Cl. CPC A61F 5/08; A61M 29/00
USPC 606/199

(57) ABSTRACT

A nasal dilator capable of introducing separating forces in the nasal outer wall tissue, has a resilient member, and a pair of spaced-apart end surfaces which can be forced toward one another from an initial flat position of the dilator to thereby substantially reduce the direct spacing therebetween by an external spacing reducing force. This results in restoring forces in the dilator tending to return it to the original direct spacing between the end surfaces. The resilient member, which is asymmetrical with respect to a centerline of the dilator that is parallel to the long axis of the dilator, has an extended surface at each end surface of the dilator which increases the adhesive surface area. The extended surface is located at each end surface of the resilient member on the side opposite the nose ala (wings) and above the alar furrow. An adhesive on the bottom surface of the resilient member adhesively engages exposed surfaces of the nasal outer wall tissues known as the lateral surface sufficiently to keep the dilator attached to the lateral wall while subjecting them to the restoring forces.

### BACKGROUND OF THE INVENTION

This invention relates to the structure of nasal dilators which reduces the cost of manufacturing and improves the performance of the dilator. Current nasal dilator designs employ one of more resilient bands which are integrated into a nasal dilator structure with flexible material that defines each end of the dilator and the intermediate section therebetween. The nasal dilator structure has sufficient length, so that the resilient band can be bent over the bridge of the nose, and each end of the band becomes adhesively attached to the soft tissue on the external wall of the nasal passage.

Bending the resilient band from its initial planar state to its deformed state with its ends in contact with the lateral walls of the nasal passages and the intermediate section of the band overlying the bridge of the nose results in forces tending to pull out on the lateral wall tissues which stabilize the walls of the nasal passages during breathing.

The present invention improves nasal dilators by simplifying the fabrication to reduce costs and improve performance. In the past nasal dilators have been designed with a resilient member which adheres to a flexible member which defines each of the ends of the dilator and the intermediate section. This flexible member is designed to increase the adhesive surface area of the dilator to enable the adhesive on the user's nose to overcome the lifting forces of the resilient member. Skin oils and moisture over time will degrade the adhesive's ability to stick to the user's nose which further requires additional adhesive surface. The addition of a flexible member increases the complexity of the dilator design and costs. The present invention is made from a single layer and has a resilient member with extended surfaces at each end which are positioned to provide optimum adhesion to the lateral wall of the nose.

The nasal dilator of the present invention is made from a resilient band which has an adhesive surface on the bottom at each end which adheres directly on the lateral wall of the nose and an intermediate section which crosses and adheres to the bridge of the nose. At each end of the resilient band there is an extended surface which is part of the resilient band which increases the adhesive surface area and enables the resilient band to adhere to the user's nose and overcome the opening forces on the lateral wall of the nose.

The nasal dilator of the present invention has a resilient band with an intermediate section which crosses the bridge of the nose and connects the two end regions. The width of the intermediate section determines the lifting force of the nasal dilator on each side of the lateral wall of the nose. The length of the intermediate section is designed to contain all of the bending of the nasal dilator when it is installed on the user's nose while the ends of the resilient band with their extended surface area are designed to adhere to the lateral wall and overcome the bending forces of the intermediate section.

Blockage of the nasal passages from swelling due to allergies, colds, and physical deformities can lead to breathing difficulty and discomfort. The nasal passages have mucus membranes which condition the air in the nasal passages prior to its arrival in the lungs. If the nasal passages are constricted due to swelling or minor deformities then the alternative is to breathe through the mouth. This means that the air bypasses the mucus membranes, losing the conditioning effects and causing irritation in the throat and lungs. At night, restrictions to breathing through the nasal passages can lead to snoring and/or sleep disturbances. In some cases, the restricted air supply can cause sleep problems brought on by a lack of oxygen.

For people with chronic blockages in the nasal passages, the alternative to correct the problem has been expensive surgery or medication. People with minor deformities and breathing problems brought on by swelling of the walls of the nasal passageways have been turning to various products fitted in or on the nose which claim to open the nasal passages.

The structure of the nose limits the options available for the design of nasal dilators. The nose terminates at the nostril, which has a slightly expanded volume immediately above it known as the vestibule. Above the vestibule, the nasal passage becomes constricted at a point called the nasal valve. At the nasal valve, the external wall of the nose consists of soft skin known as the lateral wall, which will deform with air pressure changes induced with the nasal passage during the breathing cycles. Above the nasal valve the nasal passage opens up to a cavity with turbinates over the top of the palate and turns downward to join the passage from the mouth to the throat.

The external structure of the nose consists of a skin covering over the nasal bones which are part of the skull. This gives the top of the nose a rigid structure at its base. Beyond the rigid nose bones, there is thin cartilage under the skin which is attached to the septum, which in turn contributes to the outside shape of the nose. The septum forms the wall between the two nostrils and may, if it is crooked, contribute to breathing problems.

The external surfaces of the nose have some names of the various surfaces which are important in defining how the nasal dilator of the present invention is used. At the base of the nose there are two nostrils which are separated by the septum. The septum ends at the tip of the nose which is called the apex. Above each nostril the skin on the outer wall of the nostril is referred to as the ala (wing). Also above and behind the ala, there is a crease that is the boundary of the ala and the lateral surface, as well as the cheek, called the alar furrow. Above the alar furrow is the lateral surface which covers the side of the nose between the bridge and the cheek area. On everyone's nose the lateral surface is a flat area above the alar furrow that covers nasal valve on the bottom end of the nasal bones and the thin cartilage under the skin toward the top end.

As an alternative to surgery, the structure of the nose and the current art leave two main alternatives for the design of nasal dilators. One alternative uses a tube or a similar structure which can be inserted into the nasal passage to hold it in the open position allowing the free passage of air. The disadvantage to this design is that the dilator structure covers up the mucus membranes which condition the air. Also dilators of this design are uncomfortable and can irritate the walls of the nasal passage.

The present invention is a simplified design which is very effective at opening the nasal passages and at lowering the cost of manufacturing. It is much simpler than the three layer nasal dilators that have a bottom flexible layer with adhesive which adheres to the user's nose and one or more resilient members adhering to the top of the bottom flexible layer. In addition, a second flexible layer with adhesive on the bottom adheres to the top of the resilient members as well as any top surface of the bottom flexible layer that is not covered by the resilient members. U.S. Pat. No. 5,533,503 to Doubik; U.S. Pat. Nos. 5,549,103, and 5,653,224 to Johnson; U.S. Pat. No. 6,453,901 B1 to Ierulli; and U.S. Pat. Nos. 6,478, 023 B1 and 7,114, 495 B1 to Lockwood are all examples of this type of nasal dilator design. They all consist of complex structures with many layers of costly materials and adhesives.

The present invention is also simpler than two layer nasal dilators. Two layer nasal dilators are designed with a flexible layer that defines each of the ends and intermediate section. One or more resilient bands have adhesive on the bottom side and are adhesively attached to the top surface of the flexible layer. The flexible layer has an adhesive surface on the bottom side which attaches the nasal dilator to the user's nose. U.S. Pat. No. 5,533,499 to Johnson is an example of this type of nasal dilator.

Another form of the two layer nasal dilators is a design that has a flexible layer with adhesive on the bottom side that defines each of the ends and the intermediate section of the nasal dilator. One or more resilient bands are adhesively attached to the bottom side of the flexible layer and have an adhesive on their respective bottom side. The adhesive on the bottom of the flexible layer not covered by the resilient band and the adhesive on the bottom of the resilient band hold the nasal dilator on the lateral wall of the nasal passages. U.S. Pat. Nos. 5,546,929; 5,553,605; 5,611,334; 5,718,224; 6,058,931 to Muchin as well as U.S. Pat. No. 6,098,616 to Lundy; U.S. Pat. No. 6,318,362 to Johnson; and U.S. Pat. No. 6,357,436 B1 to Kreitzer. Another variation of the two layer nasal dilator design is U.S. Pat. No. 8,342,173 B2 to Lockwood which has a cushion layer that surrounds the resilient bands in this two layer design.

There are single layer nasal dilators in the prior art that have thin tabs at each end and an intermediate section that is thicker that the tabs which define each of the end sections. In some cases the end tabs are either inserted into the nostril as taught in U.S. Pat. No. 5,479,944 to Petruson or the tabs can have adhesive on the bottom side and be adhering to the outside of the user's nose. U.S. Reissue Pat. Re 35408 to Petruson is an example of this nasal dilator which must be manufactured by a molding process. A similar nasal dilator with two ends made of thin resilient bands has a thicker intermediate section formed by an extrusion process. U.S. Pat. No. 6,029,658 to DeVoss is such an example.

There are also nasal dilators which are made from a single layer resilient band; however, they are very different from the present invention. The have end sections which are joined by an intermediate section; however, the end sections have a greater width than the intermediate section. They are also symmetrical with respect to the long axis of the dilator, and they have openings in the resilient band either at the ends and/or at the intermediate section which are designed to reduce the opening force of the resilient band. U.S. Pat. No. 5,611,333 to Johnson describes this type of nasal dilator. Another single layer nasal dilator is shown in Spanish Patent 289,561 to Iriarte which shows a resilient band of constant width that passes over the bridge of the nose and terminates at the triangular and/or alar cartilage. These single layer nasal dilator designs do not address the issue of how to achieve the best adhesion to the user's skin. The skin oils and moisture reduce the adhesive strength to the extent that a resilient band of constant width will not have enough adhesive surface to overcome the opening forces of the dilator bent over the bridge of the nose. In fact the teaching that some types of openings in the ends or intermediate section is an attempt to solve this adhesive problem. The present invention has a low cost solution to achieve the best adhesion to the user's skin.

### BRIEF SUMMARY OF THE INVENTION

An object of this invention is to provide a nasal dilator which exhibits improved performance and lower manufacturing costs relative to the nasal dilators known from the prior art.

An important feature of the present invention is to provide a basic nasal dilator which performs the function of mechanically opening the nasal passages with the simplest and most effective design. At a minimum a nasal dilator must consist of a resilient member which is long enough to pass over the bridge of the nose and have end sections which adhere to the outer walls of the nasal passages in such a way that the restoring forces in the resilient member tend to pull out on the outer walls of each nasal passage. In addition the resilient member requires that an adhesive be placed on the bottom surface of the resilient member which, when adhering to the skin on the user's nose, has sufficient strength to overcome the restoring forces of the resilient member. Most medical grade adhesives are designed to be used in applications where there are shear loads on the adhesive and unless the adhesives have a very aggressive adherence characteristics and high internal strength, they will not have the necessary adhesive strength to overcome the restoring forces of the resilient member. In addition oils and moisture from the user's skin will reduce the effective strength under tension of even the most aggressive adhesives. To insure that there is adequate adhesive strength, the nasal dilator of this present invention has extended surfaces at each end section which provide additional adhesive area.

Adding extended adhesive surfaces to each end of the resilient member is only half of the solution to the adhesive strength at each of the ends of the resilient member. On the outside of each nostril is the ala (wing). Between the ala and the lateral surface, there is the alar furrow. In order to obtain the best performance of the resilient member in a nasal dilator, the bottom edge of the resilient member should be tangent to the alar furrow. In prior art nasal dilator designs, there is usually a flexible layer designed to extend the adhesive surface with adhesive on the side facing the user's skin. The flexible layer when the dilator is properly located on the nose attempts to adhere to skin at the alar furrow and the ala. For most people the contour of the skin over the alar furrow and the ala is such that there is little effective adhesive contact by a nasal dilator in this area. In the present invention the extended adhesive surface at each end of the resilient member is on the side opposite the alar furrow on the lateral surface whose relatively flat surface increases the effective adhesion of each end section.

Another feature of the present invention is its simple design. The nasal dilator is made from a single layer of polyester which has a constant thickness throughout the structure. The polyester is normally clear, so the user's skin color shows through. In some cases, the polyester can be colored as desired and/or artwork can be applied to the extended surfaces at each of the end surfaces.

The nasal dilator in the present invention has two end surfaces and an intermediate surface therebetween. Each of the end surfaces has an extended surface area which is on the side opposite the alar furrow when the dilator is adhering to the user's nose. The intermediate surface is designed to connect each of the end surfaces over the bridge of the nose and the width of the intermediate surface determines the strength of the opening force which the nasal dilator applies to the user's nose. The length of the intermediate surface is determined to be two times the distance from the center of the bridge of the nose to a point where the end of the intermediate surface becomes parallel to the lateral surface when the intermediate surface is bent over the bridge of the nose. Each end of the intermediate surface is attached directly to each end surface respectively. The entire bottom surface of the nasal dilator has a medical grade adhesive which is designed to overcome the opening forces of the resilient member when the dilator is installed on the user's nose. The nasal dilator of the present invention also has a release liner covering the adhesive surface which has the same edge dimensions as the nasal dilator. The release liner has a slit at the middle of the intermediate surface to facilitate removal of the release liner prior to the application of the nasal dilator.

The improvements summarized above enhance the performance of the dilator and make the dilator more comfortable for the user. The improved dilator is also simpler to make as compared to the prior art.

### BRIEF DESCRIPTION OF THE DRAWINGS

The unique advantage of the present invention will become apparent to one skilled in the art upon reading the following specifications and by reference to the following drawings:

FIG. 1 is a side view of the dilator on the nose;

FIG. 2 is an exploded perspective top view of the components making up the dilator;

FIG. 3 is an exploded perspective top view of the components of an alternate design that make up the dilator.

### DETAILED DESCRIPTION OF THE INVENTION

The specific improvements provided by this invention over past nasal dilators described in the prior art are best seen in the attached drawings.

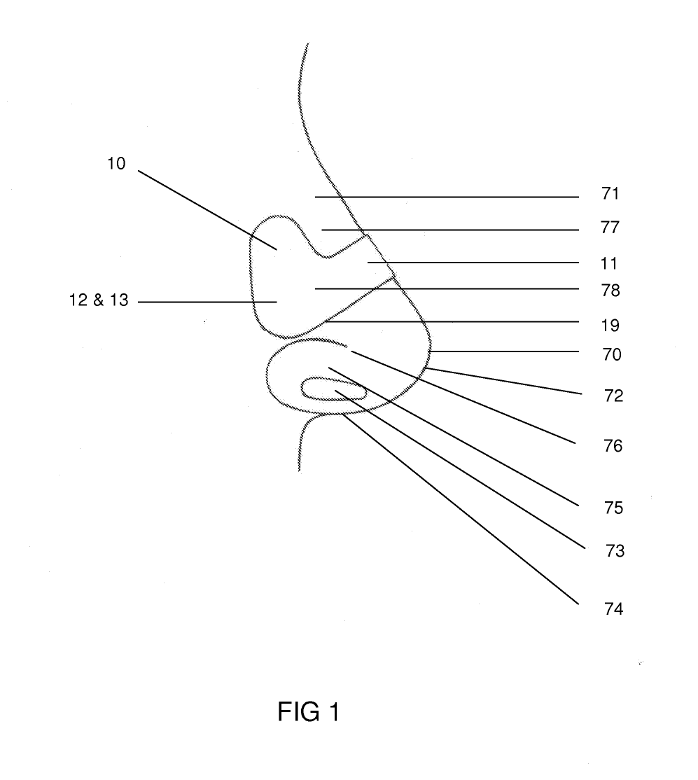

Referring to FIG. 1 the new dilator 10 is mounted on the nose 70 of the user. The nasal dilator 10 has an intermediate section 11 that is bent over the bridge 71 of the nose 70 and each end 12 and 13 of the nasal dilator 10 is positioned over the lateral surface 77 of the nose 70.

The structure of the nose 70 is important to demonstrate the unique advantages of the nasal dilator 10 in this invention. Starting at the tip of the nose 70 we have the apex 72 and at the bottom of the nose 70 and between the two nostrils 73 we have the septum 74. Above and on the outside surface of each nostril 73, there is a surface known as the ala (wing) 75. The boundary of the ala 75 and the lateral surface 77 is the alar furrow 76 which is a heavily contoured demarcation. The lateral surface 77 is on each side of the nose 70 and is a flat surface that makes up the side of the nose 70 from the alar furrow 76 up to just below the user's eye.

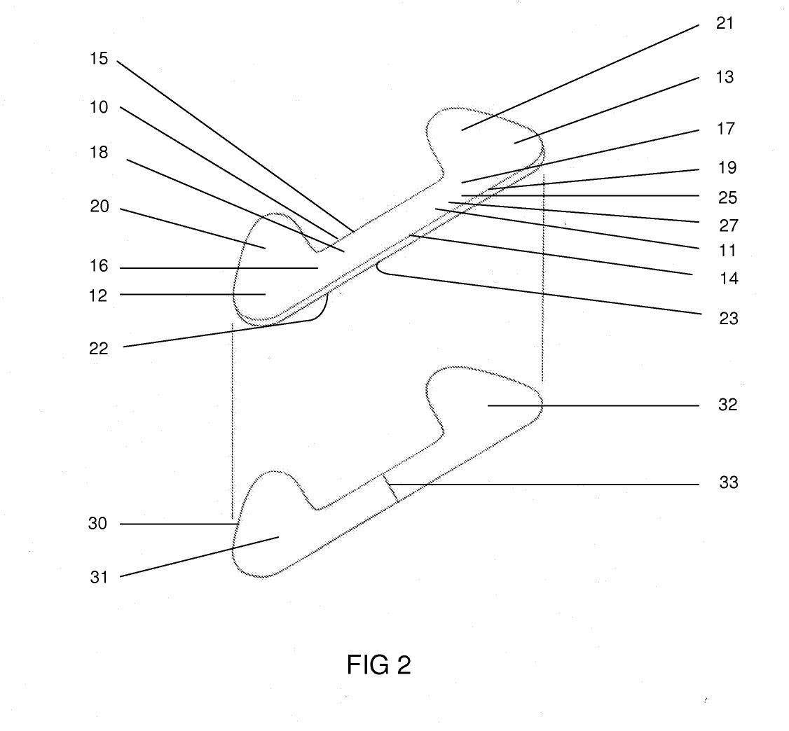

Referring to FIG. 2 the new dilator 10 is fabricated from a polyester film forming a resilient member 25. The polyester film has a constant thickness over the entire surface of the dilator 10. The dilator 10 thickness is selected to achieve the desired opening force on the lateral surface 77 of the nose 70.

The new dilator 10 consists of two ends 12 and 13 which are connected by an intermediate section 11. The intermediate section 11 has a front edge 14 and a back edge 15. The width of the intermediate section 11 between the front edge 14 and the back edge 15 determines the opening strength of the dilator 10. The intermediate section has a length between each of the dilator ends 12 and 13. The ends 16 and 17 of the intermediate section 11 are determined to be the points where the ends 16 and 17 of the intermediate section 11 are parallel to the lateral surface 77 when the dilator is bent over the bridge 71 of the nose 70.

At each end 16 and 17 of the intermediate section 11 the width of the dilator 10 increases forming extended surfaces 20 and 21 on the respective ends 12 and 13 of the dilator 10. The extended surfaces 20 and 21 are on the top side 18 of the dilator 10 when it is mounted on the user's nose 70. On the bottom surface 22 of the dilator 10, there is a medical grade adhesive 23 which enables the dilator 10 to adhere to the lateral surface 77 of the user's nose 70. The adhesive 23 covers the entire bottom surface 22 of the dilator 10 and has sufficient strength to overcome the forces generated when the resilient member 25 is deformed as it is bent over the bridge 71 of the nose 70.

The extended surfaces 20 and 21 are necessary to increase the adhesive contact surface at each end 12 and 13 of the dilator 10 due to the effects of skin oils and moisture which over time will degrade the strength of the medical grade adhesive 23. The extended surfaces 20 and 21 are located on one side of the longitudinal centerline of the dilator 10 creating an asymmetrical configuration of the dilator 10.

The front edge 14 of the dilator 10 is also the bottom side 19 of the extended surface 20 and 21 where it extends beyond the ends 16 and 17 of the intermediate section 11. When the dilator 10 is installed on the user's nose 70 the bottom side 19 of the extended surface 20 and 21 is designed to be tangent to and above the alar furrow 76 of the user's nose 70. This positions the ends 16 and 17 of the intermediate section 11 almost directly over the nasal valve 78 which makes the dilator very effective. Prior designs of nasal dilators have extended adhesive surfaces which are made from flexible fabrics or films which attempt to adhere to the ala (wing) 75 or alar furrow 76 surfaces which are heavily contoured thus reducing the effectiveness of the adhesive surfaces. In contrast to the prior art the new dilator 10 is designed to adhere to the lateral surface 77 which is flat and insures that the adhesive 23 will adhere to the user's nose for an extended period.

In order to protect the adhesive 23 surface during manufacturing and prior to use, a release liner 30 has two segments 31 and 32 which meet at the center slit 33 of the intermediate section 11 of the dilator 10. The center slit 33 makes it easy for the user to remove the release liner 30 segments 31 and 32 prior to installing the dilator 10. The release line 30 is the same shape as the nasal dilator 10 to prevent overstrike issues during manufacturing.

The resilient member 25 of the new dilator 10 is fabricated from a polyester sheet 27 that has a constant thickness over the entire surface. The thickness of the polyester sheet 27 can fall in a range from 0.008 inches up to 0.015 inches depending on the desired opening force of the dilator 10. The constant thickness polyester sheet 27 allows the dilator 10 to be manufactured in a converting process using rotary cutters.

The resilient member 25 is normally clear polyester sheet 27 that can have either a shiny or matte finish. This design allows the user's skin color to be seen through the nasal dilator 10. In some cases the polyester sheet 27 may be tinted or colored to meet special applications. One such application would use matte black polyester sheet 27 for nasal dilators used in sports.

The new nasal dilator 10 can also have an additional layer of flexible material (not shown) laminated to the top surface 18 of the resilient member 25 to add color, to allow for printing, to accommodate a logo, or to add artistic designs. The flexible material would be cut to the same perimeter as the resilient member 25, but would not affect the performance of the nasal dilator 10.

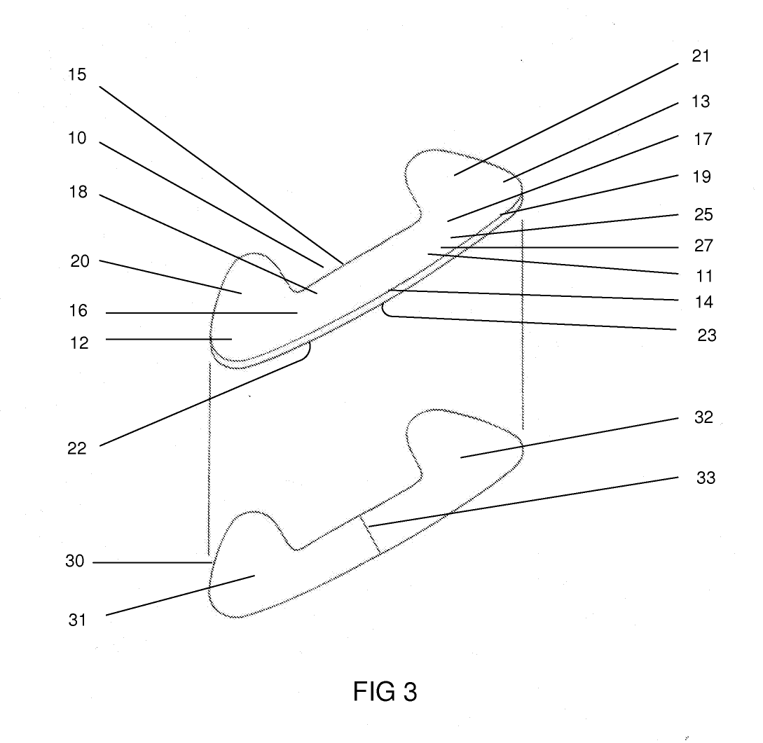

Referring to FIG. 3 the new dilator 10 is shown in an alternate embodiment which allows the resilient member 25 to be located closer to the apex 72 of the nose 70 and still allow the bottom side 19 of the extended surface 20 and 21 to be tangent to and above the alar furrow 76 of the user's nose 70.

The new dilator 10 consists of two end 12 and 13 which are connected by an intermediate section 11. The intermediate section 11 has a front edge 14 and a back edge 15. The width of the intermediate section 11 between the front edge 14 and the back edge 15 determines the opening strength of the dilator 10. In this configuration the width of the intermediate section 11 at the center of the nasal dilator 10 is greater than the width at each end 16 and 17 of the intermediate section 11. This is achieved by adding a curvature to the bottom side 19 of the resilient member 25 that extends from the center of the intermediate section 11 to each end 12 and 13 of the nasal dilator 10. Adjusting the radius of curvature of the bottom side 19 will alter the opening force of the nasal dilator and allow the resilient member 25 to be positioned closer to the apex 72 of the nose 70.

The ends 16 and 17 of the intermediate section 11 are determined to be the points where the ends 16 and 17 of the intermediate section 11 are parallel to the lateral surface 77 when the dilator is bent over the bridge 17 of the

What is claimed is:

1. A nasal dilator capable of introducing separating stresses in nasal outer wall tissues and having a pair of spaced-apart end surfaces which, if forced toward one another from initial positions when the resilient member is flat to substantially reduce direct spacing therebetween by a spacing reducing force external to said resilient member results in restoring forces in said resilient member tending to restore said direct spacing between said end surfaces and comprising:
a resilient member having a constant thickness which is asymmetrical along the long axis of the nasal dilator with extended surfaces at each end which adhere to the lateral surface of the nose and have opposite sides which are tangent to the alar furrow of the nose; and
an engagement means adhered to said end surfaces and capable of engaging exposed surfaces of nasal outer wall tissues sufficiently to remain so engaged against said restoring forces.

2. A nasal dilator according to claim 1 wherein the engagement means is a medical grade adhesive.

3. A nasal dilator according to claim 1 wherein the thickness of the dilator is constant over the entire surface.

4. A nasal dilator according to claim 1 wherein the resilient member is fabricated from transparent materials.

5. A nasal dilator according to claim 1 wherein the resilient member is fabricated from colored materials.

6. A nasal dilator according to claim 1 wherein the resilient member is covered by a layer that is colored, is printed, has a logo, or an artistic design.

7. A nasal dilator according to claim 1 including a release liner protecting the adhesive on the bottom surface of the resilient member and is cut to the same perimeter.

8. A nasal dilator capable of introducing separating stresses in nasal outer wall tissues and having a pair of spaced-apart end surfaces which, if forced toward one another from initial positions when the resilient member is flat to substantially reduce direct spacing therebetween by a spacing reducing force external to said resilient member results in restoring forces in said resilient member tending to restore said direct spacing between said end surfaces and comprising:
a resilient member having a constant thickness which is asymmetrical along the long axis of the nasal dilator with extended surfaces at each end which adhere to the lateral surface of the nose and have opposite sides which are tangent to the alar furrow of the nose;
an intermediate section of the resilient member which is configured in part so that the width is greatest in the center and diminishes towards each end;
an engagement means adhered to said end surfaces and capable of engaging exposed surfaces of nasal outer wall tissues sufficiently to remain so engaged against said restoring forces.

9. A nasal dilator according to claim 8 wherein the engagement means is a medical grade adhesive.

10. A nasal dilator according to claim 8 wherein the thickness of the dilator is constant over the entire surface.

11. A nasal dilator according to claim 8 wherein the resilient member is fabricated from transparent materials.

12. A nasal dilator according to claim 8 wherein the resilient member is fabricated from colored materials.

13. A nasal dilator according to claim 8 wherein the resilient member is covered by a layer that is colored, is printed, has a logo, or an artistic design.

14. A nasal dilator according to claim 8 including a release liner protecting the adhesive on the bottom surface of the resilient member and is cut to the same perimeter.

15. A nasal dilator for preventing outer wall tissue of nasal passages of a nose from drawing in during breathing, the dilator having a constant thickness, a longitudinal axis, opposite end surfaces, an intermediate section, and a normally substantially planar state, the dilator comprising:
an elongated resilient member which is asymmetrical along the longitudinal axis of the nasal dilator with extended surfaces at each end which are designed to adhere to the lateral surface of the nose and have opposite sides which are designed to be tangent to the alar furrow when installed on the nose; and
an engagement means adhered to said end surfaces and capable of engaging exposed surfaces of nasal outer wall tissues.

16. A nasal dilator according to claim 15 wherein the engagement means is a medical grade adhesive.

17. A nasal dilator according to claim 15 wherein the thickness of the dilator is constant over the entire surface.

18. A nasal dilator according to claim 15 wherein the resilient member is fabricated from transparent materials.

19. A nasal dilator according to claim 15 wherein the resilient member is fabricated from colored materials.

20. A nasal dilator according to claim 15 wherein the resilient member is covered by a layer that is colored, is printed, has a logo, or an artistic design.

21. A nasal dilator according to claim 15 including a release liner protecting the adhesive on the bottom surface of the resilient member and is cut to the same perimeter.

22. A nasal dilator for preventing outer wall tissue of nasal passages of a nose from drawing in during breathing, the dilator having a constant thickness, a longitudinal axis, opposite end surfaces, an intermediate section, and a normally substantially planar state, the dilator comprising;
an elongated resilient member which is asymmetrical along the longitudinal axis of the nasal dilator with extended surfaces at each end which are designed to adhere to the lateral surface of the nose and have opposite sides which are designed to be tangent to the alar furrow when installed on the nose;
an intermediate section of the resilient member which is configured in part so that the width is greatest in the center and diminishes towards each end; and
an engagement means adhered to said end surfaces and capable of engaging exposed surfaces of nasal outer wall tissues.

23. A nasal dilator according to claim 22 wherein the engagement means is a medical grade adhesive.

24. A nasal dilator according to claim 22 wherein the thickness of the dilator is constant over the entire surface.

25. A nasal dilator according to claim 22 wherein the resilient member is fabricated from transparent materials.

26. A nasal dilator according to claim 22 wherein the resilient member is fabricated from colored materials.

27. A nasal dilator according to claim 22 wherein the resilient member is covered by a layer that is colored, is printed, has a logo, or an artistic design.

28. A nasal dilator according to claim 22 including a release liner protecting the adhesive on the bottom surface of the resilient member and is cut to the same perimeter.
